(19) United States
(12) Patent Application Publication
(10) Pub. No.: US 2012/0203175 A1
(43) Pub. Date: Aug. 9, 2012

(54) OSTOMY SYSTEM

(76) Inventor: Yifeng Sun, Shanghai (CN)

(21) Appl. No.: 13/141,242

(22) PCT Filed: Sep. 25, 2010
(86) PCT No.: PCT/CN10/77256

§ 371 Date: Jun. 21, 2011

(30) Foreign Application Priority Data
Jul. 9, 2010 (CN) 201010226288.6

(51) Int. Cl. A61M 29/02

(52) U.S. Cl. 604/103.09; 604/96.01; 604/103.1

(57) ABSTRACT

An illustrative system includes a catheter with a first conduit having a first distal port. The first distal port is in fluid communication with an interior of a balloon mounted to the catheter. The catheter also includes a second conduit having a second distal port, where the second distal port is configured to receive a needle. The balloon is configured to inflate responsive to a fluid received through the first distal port such that the needle is suitably positioned to penetrate an external surface of a subject.

### CROSS-REFERENCE TO RELATED APPLICATIONS

The present application is a U.S. national stage application claiming the benefit of International Application No. PCT/CN2010/077256, filed on Sep. 25, 2010, which claims the benefit of Chinese Application No. 201010226288.6, filed on Jul. 9, 2010, the entire contents of which are incorporated herein by reference in their entireties.

### BACKGROUND

The following description is provided to assist the understanding of the reader. None of the information provided or references cited is admitted to be prior art.

Ostomy can generally refer to any procedure for creating an opening which connects a portion of a body cavity to the outside environment. An ostomy can be used to discharge waste from the body. An ostomy can also be used to feed a subject. As an example, there are many esophageal subjects who are unable to eat because of advanced esophageal cancer, esophageal cancer recurrence, and other medical conditions. One method to feed such subjects is to provide them with a gastrostomy or jejunostomy feeding tube. Traditional feeding tube insertion procedures include percutaneously inserting the feeding tube with the assistance of an endoscope. Feeding tubes can also be inserted radiologically and/or surgically. The insertion procedure utilized for a given subject can depend on the severity of the subject's esophageal ailment, the surgery history of the subject, other medical conditions affecting the subject, etc.

### SUMMARY

An illustrative system includes a catheter with a first conduit having a first distal port. The first distal port is in fluid communication with an interior of a balloon mounted to the catheter. The catheter also includes a second conduit having a second distal port, where the second distal port is configured to receive a needle. The balloon is configured to inflate responsive to a fluid received through the first distal port such that the needle is suitably positioned to penetrate an external surface of a subject.

An illustrative process includes inserting a catheter into a cavity of a subject. The catheter includes a first conduit having a first distal port in fluid communication with an interior of a balloon mounted to the catheter. A second conduit of the catheter includes a second distal port. The balloon is inflated to suitably position a needle to penetrate an external surface of the subject. The needle is protruded through the second distal port to puncture the external surface of the subject.

Another illustrative system includes a catheter and a stent mounted to the catheter. The catheter includes a first conduit having a first distal port. The stent is mounted to the catheter so that the stent is within an interior of a balloon mounted to the catheter. The stent is expandable to maintain a position of a cavity wall of a subject after the balloon at least partially deflates.

Another illustrative process includes inserting a catheter into a cavity of a subject. The catheter includes a first conduit having a first distal port in fluid communication with an interior of a balloon mounted to the catheter. The balloon is inflated to position a cavity wall of the cavity adjacent to an abdominal wall of the subject. A stent is expanded to maintain a position of the cavity wall after the balloon at least partially deflates. The stent is mounted to the catheter so that the stent is within the interior of the balloon.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1A is a complete view of an illustrative embodiment of a catheter system.

FIG. 1B is a cross-sectional view of a catheter of the catheter system in FIG. 1A in accordance with an illustrative embodiment.

FIG. 1C is a partial view of the catheter system in FIG. 1A with an inflated balloon in accordance with an illustrative embodiment.

FIG. 2 illustrates an orientation of a distal needle port relative to an abdominal wall in accordance with an illustrative embodiment.

FIG. 3 is a flow diagram illustrating a process for performing a transluminal ostomy in accordance with an illustrative embodiment.

FIG. 4A is a side view of a catheter system with a stent in a compressed state in accordance with an illustrative embodiment.

FIG. 4B is a side view of the catheter system of FIG. 4A with the stent in an expanded state in accordance with an illustrative embodiment.

FIG. 5A illustrates a catheter system with a stent in a compressed state in accordance with a second illustrative embodiment.

FIG. 5B illustrates the catheter system of FIG. 5A with the stent in an expanded state in accordance with an illustrative embodiment.

FIG. 6 is a flow diagram illustrating a process for performing a percutaneous ostomy in accordance with an illustrative embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

Described herein are illustrative systems (such as but not limited to catheter systems) and methods for use in performing a gastrostomy, jejunostomy, cystostomy, and/or any other ostomy procedure on a subject. The term “subject” should be broadly construed to include human subjects as well as animal subjects of all kinds such as, but not limited to, domesticated animals, zoo animals, farm animals, wild animals, endangered animals, race animals, working animals, pets, and aquatic animals. Illustrative examples of animals may include, but are not limited to, mammals, such as, but not limited to, canines (e.g., dogs), felines (e.g., cats), and equines (e.g., horses), birds, amphibians, reptiles, and other animal subjects. As used herein, a system (or catheter system) can refer to an assembly that includes a catheter. In an illustrative embodiment, the system (or catheter system) can be used to perform an ostomy. The catheter can have a distal end that is inserted through a mouth or other opening into a cavity of the subject. The cavity can be a stomach (or gastric cavity), jejunal lumen, intestine, bladder, and/or any other applicable cavity through which an ostomy is to be performed. The catheter can also have a proximal end opposite the distal end that will remain outside of the subject and be manipulated by the user when the system is inserted into the cavity of the subject. It will be readily understood by those of skill in the art that the term “proximal” and “distal” as used herein in connection with “end”, “port” or the like in describing various components of the system are typically designated based on their positions relative to the user when the system is in operation. For example, an end or port that is at a greater distance from the user may be called “distal” as compared with an end or port that is closer to the user when the system is being used to perform an ostomy. The catheter can have a substantially circular or oval cross-sectional shape. Alternatively, other cross-sectional shapes may also be used, such as, but not limited to, polygonal shapes (e.g. triangles, squares, rectangles, hexagons, or trapezoids, etc.) with round contour, irregular cross-sectional shapes and cross-sectional shapes that include combinations of curved and straight lines. Illustratively, the catheter can have a diameter of between approximately 1.5 millimeters (mm) and 3 mm, between approximately 1 mm and 3.5 mm, or between approximately 0.5 mm and 4 mm, e.g., approximately 0.5 mm, 1 mm, 1.5 mm, 2 mm, 2.5 mm, 3 mm, 3.5 mm, 4 mm, etc. Illustratively, the catheter can have a length of between approximately 100 centimeters (cm) and 150 cm, between approximately 90 cm and 160 cm, or between approximately 80 cm and 170 cm, e.g., approximately 100 cm, 110 cm, 120 cm, 130 cm, 140 cm, 150 cm, 160 cm, 170 cm, etc. Alternatively, other diameters and/or lengths may be used as appropriate for e.g., the size, condition of the subject and/or the size, location of the cavity. The diameters and/or lengths of the catheter can be determined as known to those of skill in the art. The catheter can be constructed of silicone, silicon rubber, polyvinyl chloride, polyurethane, other polymers and copolymers, and/or any other suitable biocompatible materials known to those of skill in the art.

The system (or catheter system) can also include a balloon mounted to the distal end of the catheter. The balloon is intended to be placed within the cavity of the subject. The balloon can be mounted to the catheter such that at least a portion of the catheter runs through at least part of an interior of the balloon. As such, the balloon can have a point of entry (e.g., hole) for the catheter and optionally a point of exit (e.g., hole) for the catheter. The point of entry and the point of exit can have the same diameter as the catheter, a smaller diameter than the catheter, or a larger diameter than the catheter depending on the embodiment. The balloon can be mounted to the catheter at the point of entry and the point of exit using an adhesive, using one or more fasteners, and/or by any other suitable method. As used herein, the term “mount” can include join, unite, connect, associate, insert, hang, hold, affix, attach, fasten, bind, paste, secure, bolt, nail, glue, screw, rivet, solder, weld, and other like terms. In one embodiment, the balloon can have a length of approximately 1 cm to 15 cm, approximately 2 cm to 12 cm, or approximately 3 cm to 10 cm, e.g. approximately 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, etc., and a maximum inflated diameter of between approximately 5 cm and 15 cm, between approximately 6 cm and 14 cm, or between approximately 8 cm and 12 cm, e.g. approximately 5 cm, 6 cm, 7 cm, 8 cm, 9 cm, 10 cm, 11 cm, 12 cm, 13 cm, 14 cm, 15 cm, etc. Alternatively, the diameter of the balloon when inflated can be between approximately 4 cm and 16 cm, 3 cm and 17 cm, etc. Alternatively, other lengths and diameters may be used depending on e.g., the size of the subject, the size of the cavity, the type of cavity, etc. The diameters and/or lengths of the balloon can be determined as known to those of skill in the art. The balloon can be made from polyethylene, polyvinyl chloride, other polymers and copolymers, silicone, rubber, latex, and/or any other biocompatible materials known to those of skill in the art.

The catheter can also include a first conduit that includes a first distal port and a first proximal port. The first distal port and the first proximal port can be mounted to the first conduit or can be extensions of the first conduit. The first conduit can be integrally formed as part of the catheter and made from the same material(s) as the catheter. Alternatively, the first conduit can be formed separately from the catheter and assembled into the catheter. The first conduit can have a uniform or varying diameter, depending on the embodiment. The first conduit can also be the same length as, longer than, or shorter than the catheter, depending on the embodiment. In some embodiments, the first conduit is in fluid communication with the interior of the balloon through the first distal port. For example, the balloon may be inflated in response to a fluid introduced from the first proximal port, through the first conduit, out of the first distal port and into the balloon. In these embodiments, the first conduit may be referred to as a balloon conduit, and the first distal port and first proximal port may be referred to as a balloon distal port and a balloon proximal port, respectively. In one embodiment, the balloon can be integral to the first conduit, e.g. as extension from the first distal port, and mounted to the catheter. Alternatively, the balloon may be formed separately and mounted to the distal end of the catheter as described above. In some embodiments, the first distal port (or distal balloon port) can be one or more holes or openings through which a fluid can be introduced into balloon. In use, a fluid can be introduced into the first proximal port. The fluid can travel through the first conduit and out of the first distal port to inflate the balloon. The fluid can be any type of biocompatible gas or liquid, such as, but not limited to, air, an inert gas, water, saline solution, etc.

The catheter can also include a second conduit. In some embodiments, the second conduit is configured for the passage of a needle and may be referred to as a needle conduit. The needle conduit can be cylindrical in shape and can be made from any suitable biocompatible material(s) known to those of skill in the art. Alternatively, the needle conduit can be any other suitable shape. The needle conduit can be formed integrally as part of the catheter, and can be made from the same material as the catheter. Alternatively, the needle conduit can be formed separately from the catheter and assembled into the catheter. In one embodiment, the needle conduit can be approximately the same length as the catheter. Alternatively, the needle conduit can be longer or shorter than the catheter. In general, the diameter of the needle conduit can be large enough to allow passage of a needle, but smaller than the overall diameter of the catheter less the diameter of the balloon conduit (or first conduit). The needle conduit can be uniform in diameter or may have a varying diameter, depending on the embodiment.

The needle conduit (or second conduit) can include a second distal port (or distal needle port) and a second proximal port (or proximal needle port). The second distal port and the second proximal port can be mounted to the needle conduit or can be extensions of the needle conduit. The second distal port can be mounted to the balloon such that inflation of the balloon causes the second distal port and at least a portion of the needle conduit to bend (or deflect) away from a central axis of the catheter. In an illustrative embodiment, the second distal port can be mounted to the balloon such that a needle can be protruded from the second distal port without puncturing the balloon. As an example, the second distal port can be mounted in an air tight fashion within a hole in a surface of the balloon such that the perimeter of the hole in the surface of the balloon forms an air tight seal about the needle conduit and/or the second distal port. As such, the balloon can be inflated without fluid escaping through the hole in the surface of the balloon.

In some embodiments, the system may be inserted into a cavity of a subject with the assistance of a guide wire. Illustratively, a guide wire can be inserted into the cavity of the subject, and the system can then be inserted into the cavity of the subject by running the catheter over the guide wire. In one embodiment, the catheter can also include a third conduit (or guide wire conduit) having a third distal port (or distal guide wire port) and a third proximal port (or proximal guide wire port) for the passage of the guide wire. Alternatively, the needle conduit (or second conduit) may serve as a guide wire conduit. The guide wire can be made of any suitable material, such as, but not limited to, stainless steel, other metals/metal alloys, and/or polymers. The guide wire can be of any suitable size and configuration known to those of skill in the art.

In an illustrative embodiment, the balloon can be inflated within the cavity of the subject to suitably position the needle to penetrate an external surface (or outer skin) of the subject. In some embodiments, the external surface (or outer skin) is the external surface (or outer skin) of an abdominal wall of the subject. During this process, the balloon may be at least partially deflated and/or re-inflated as appropriate. Suitably positioning the needle can refer to aligning the needle such that the needle penetrates an abdominal wall of the subject at approximately a ninety degree angle. Suitably positioning the needle can also refer to positioning a plane that contains at least a portion of an outer surface of the second distal port parallel to a portion of the abdominal wall of the subject that is adjacent to the outer surface of the second distal port. The catheter, the balloon, the second distal port, and/or the needle can be positioned using one or more radio-opaque markers mounted to any component of the system along with x-ray images that identify the radio-opaque markers within the subject. The system may also be positioned using one or more magnets mounted to the system, using one or more protuberances mounted to the system, or by other methods that are known or will become apparent to those of skill in the art in light of the present disclosure. For illustrative purpose only, some embodiments are described in more detail below with reference to the figures.

Once the second distal port and the needle are suitably positioned, a user can protrude the needle through the external surface (or outer skin) of the subject. The needle, which can have a sharpened distal end for piercing tissue, can have a diameter such that the needle fits within the needle conduit. The needle can have a length that is the same as, shorter than, or longer than the length of the catheter. In one embodiment, the needle can be controlled using a plunger. In such an embodiment, the plunger can be mounted to a first end (or proximal end) of a rigid or semi-rigid wire and the needle can be mounted to a second end (or distal end) of the wire. The user can depress the plunger, which can be mounted to the second proximal port, to protrude the needle through the external surface of the subject. Alternatively, a plunger may not be used and the user can manually protrude the needle through the second distal port and out through the external surface of the subject by manipulating the needle or a rigid or semi-rigid wire mounted to the needle.

In one embodiment, once the needle penetrates the external surface of the subject, a medical tube can be placed within the cavity of the subject using established procedures well known to those of skill in the art. Further description about some illustrative embodiments of medical tube insertion is given below. Alternatively, a medical tube may not be used. Upon completion of the ostomy and optionally medical tube insertion procedure, the balloon can be deflated by at least partially withdrawing the liquid, and the system can be removed from the subject.

In another embodiment of a system (or catheter system) provided in this disclosure, a catheter can have substantially the same dimensions and be made from the same materials as the above-described catheter. An expandable stent can be mounted to the distal end of the catheter, and a balloon can also be mounted to the distal end of the catheter such that the balloon surrounds the expandable stent. The balloon can have the same dimensions and be made from the same materials as the above-described balloon. The catheter can include a first conduit (or balloon conduit) having a first distal end and a first proximal end. The first conduit can have the same dimensions and/or configuration as the above-described first conduit. The first conduit can be configured to receive a fluid for inflating the balloon as described above.

In certain embodiments, the expandable stent (or stent) can include a plurality of flexible wires made from a biocompatible metal/metal alloy or other biocompatible material used for stents. The wires may be between approximately 8 and 15 cm, between approximately 7 and 16 cm, or between approximately 6 cm and 17 cm, e.g., approximately 6 cm, 7 cm, 8 cm, 9 cm, 10 cm, 11 cm, 12 cm, 13 cm, 14 cm, 15 cm, 16 cm, 17 cm, etc. in length. The stent, when expanded, may have a diameter of between approximately 5 and 10 cm, between approximately 4 cm and 11 cm, or between approximately 3 and 12 cm, e.g., approximately 3 cm, 4 cm, 5 cm, 6 cm, 7 cm, 8 cm, 9 cm, 10 cm, 11 cm, 12 cm, etc. Alternatively, any other length and/or diameter may be used based on e.g. the size of the subject, the size of the cavity, etc.

In one embodiment, one end of each of the wires can be mounted to a cap and/or to the center wire of the stent, and the other end of each of the wires can be mounted to the catheter and/or to the balloon conduit, and the stent can be expanded using the cap/center wire of the stent. The center wire can be more rigid than the plurality of wires of the stent, and can have a larger diameter than the plurality of wires of the stent. The center wire can be made from the same material as the wires of the stent or from a different material, depending on the embodiment. The center wire, which can be mounted to the cap of the stent, can be pulled outward from a proximal end of the catheter to draw the cap towards the catheter and expand the stent. A user can use a center wire handle mounted to the center wire to pull the center wire outward to expand the stent and to push the center wire inward to compress the stent. The center wire can run through a stent conduit (or second conduit) of the catheter that includes a proximal stent port (or opening) and a distal stent port (or opening). The stent conduit can have a diameter that is sufficient to receive the center wire, and can have a length that is greater than, less than, or equal to the length of the catheter.

Alternatively, the expandable stent (or stent) may not include a center wire. In such an embodiment, a sheath can be slidably mounted to the catheter and used to expand/compress the stent. The sheath (or sleeve) can be made from a biocompatible material having enough strength to contain the stent. The sheath can be shorter in length than the catheter and can have an interior diameter that is larger than or approximately the same size as the exterior diameter of the catheter. The interior diameter of the sheath can be large enough such that sheath can maintain the stent in a compressed state when the sheath is slid along the catheter and over the stent. Sliding the sheath toward the distal end of the catheter and over the stent can compress the plurality of wires of the stent. Sliding the sheath toward the proximal end of the catheter such that the sheath is no longer covering the stent can allow and/or cause the flexible wires of the stent to expand.

It will be understood that the above embodiments are given as illustrative examples only and that other configuration of the stent will be apparent to those of skill in the art in light of the present disclosure.

In an illustrative embodiment, the system (or catheter system) can be positioned (by any of the methods described above) such that the stent and the balloon are within a cavity of a subject. In one embodiment the catheter can be inserted with the assistance of a guide wire as described above and the catheter can be constructed accordingly. The stent can be in the compressed state and the balloon can be deflated when the system is inserted into the cavity. The balloon can be inflated by introducing a fluid into the balloon. Inflation of the balloon can position a cavity wall (such as a stomach wall, etc.) adjacent (or proximate) to an abdominal wall of the subject. The stent can be expanded e.g. by manipulating the center wire or the sheath, depending on the embodiment. The stent can be expanded before, during, or after inflation of the balloon, depending on the embodiment. A user can introduce a needle through an external surface (or outer skin) of the subject, through the abdominal wall, through the cavity wall, through the balloon, and into the cavity. The balloon may at least partially deflate as a result of being punctured by the needle, yet the expanded stent can be used to substantially maintain the position of the cavity wall relative to the abdominal wall. In one embodiment, a medical tube can be run (or extended) over the needle so that at least a portion of the medical tube is within the cavity. Other procedures of placing a medical tube are well known to those of skill in the art. Alternatively, a medical tube may not be used. Upon completion of the ostomy and optionally medical tube insertion procedure, the stent can be compressed e.g. by manipulation of the center wire or the sheath, and the system can be removed from the subject.

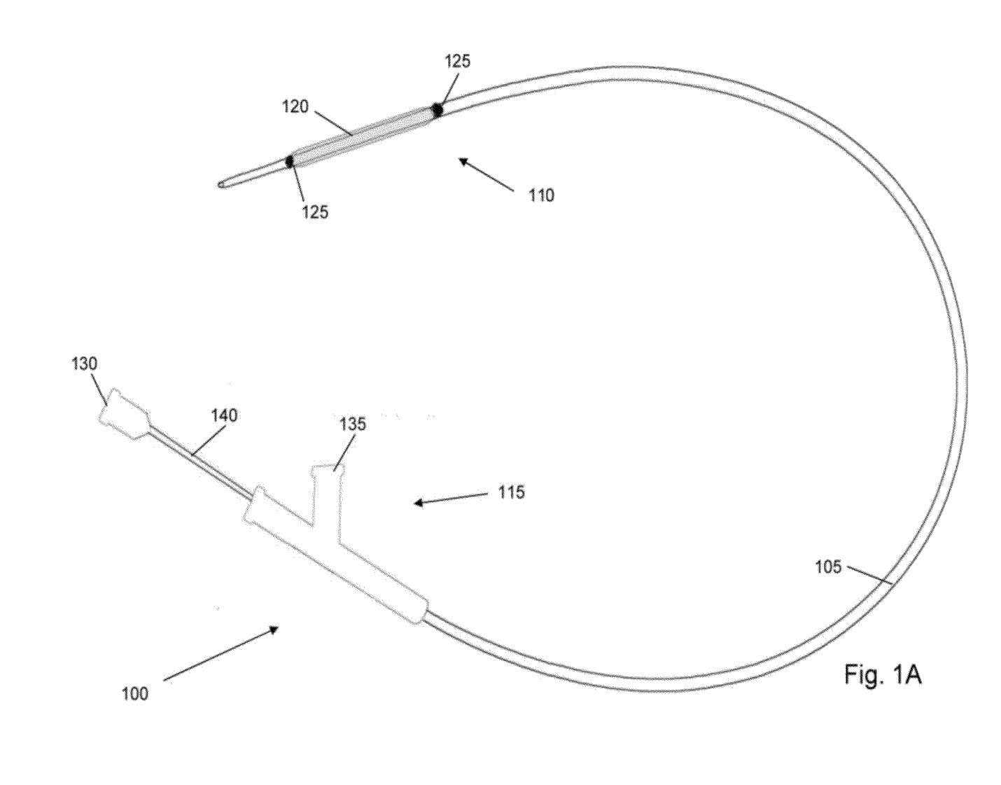

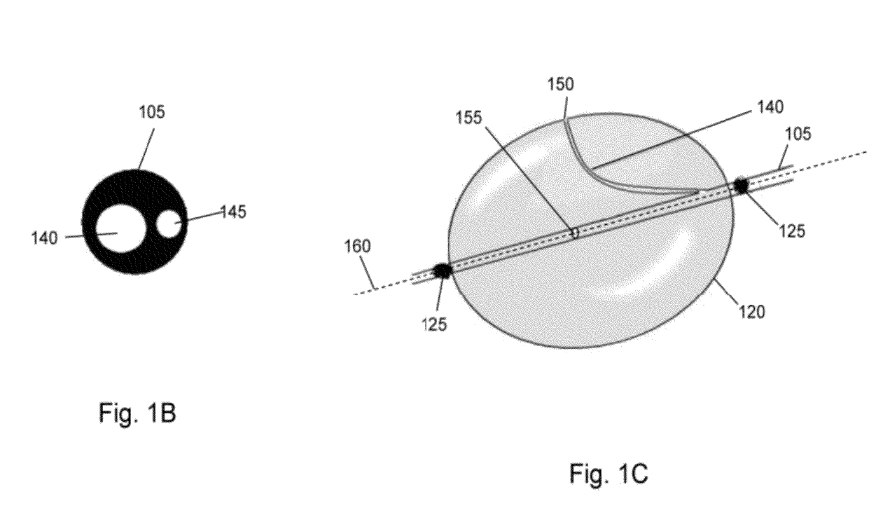

Additional details and embodiments are described with reference to the figures. FIGS. 1A-1C illustrate a system (or catheter system) 100 in accordance with an illustrative embodiment. FIG. 1A is a complete view of catheter system 100 in accordance with an illustrative embodiment. FIG. 1B is a cross-sectional view of a catheter 105 of catheter system 100 in accordance with an illustrative embodiment. FIG. 1C is a partial view of catheter system 100 with a balloon 120 inflated in accordance with an illustrative embodiment. Catheter system 100 can be used to perform a transluminal gastrostomy, transluminal jejunostomy, and/or any other applicable ostomy procedure on a subject.

Catheter 105 of catheter system 100 has a distal end 110 and a proximal end 115. In use, distal end 110 of catheter 105 can be inserted into a cavity of a subject. The cavity can be a stomach (or gastric cavity), jejunal lumen, intestine, bladder, and/or any other applicable cavity through which an ostomy is to be performed.

Catheter system 100 can be introduced through the mouth or other opening of the subject and into the cavity. In one embodiment, a guide wire may be used to insert catheter system 100 into the cavity. Any type of guide wire known to those of skill in the art may be used. As an example, the guide wire may be inserted through the mouth or other opening and into the cavity such that a proximal end of the guide wire remains external to the subject. A physician, veterinarian, or other user can place the proximal end of the guide wire into a distal guide wire port (not shown) of catheter 105, and insert catheter 105 into the cavity such that the guide wire is run through a guide wire conduit (not shown) in communication with the distal guide wire port and out of a proximal guide wire port (not shown) that is also in communication with the guide wire conduit. The distal guide wire port can be any type of port (or opening) in distal end 110 of catheter 105 that is configured to receive the guide wire. Similarly, the proximal guide wire port can be any type of port (or opening) in proximal end 115 of catheter 105 that is configured to receive the guide wire. The guide wire conduit can be any type of conduit (or tube) that is mounted to both the proximal guide wire port and the distal guide wire port and that is configured to receive the guide wire. The proximal guide wire port and/or the distal guide wire port may also be extensions of the guide wire conduit. Any configuration of the guide wire ports and guide wire conduit known to those of skill in the art may be used. Alternatively, a needle conduit as described below may serve as a guide wire conduit. In an alternative embodiment, catheter system 100 may be placed based on visual observation of a length of catheter 105 that remains outside of the subject as will be understood by those of skill in the art. Catheter system 100 may also be placed by touching of the abdominal wall to feel catheter system 100. As an example in which the cavity is a stomach, a physician, veterinarian, or other user can introduce catheter system 100 into a mouth or other opening of the subject. The user can place his/her hand(s) on the subject's skin (i.e., the skin external to the cavity of the subject) to feel a position of catheter system 100 and to determine how far catheter system 100 is to be inserted into the subject.

Balloon 120 is mounted to distal end 110 of catheter 105. As illustrated with reference to FIG. 1A, a portion of catheter 105 can run through an interior of balloon 120 such that balloon 120 has a point of entry (of catheter 105) and a point of exit (of catheter 105). The point of entry and the point of exit can be holes in balloon 120 that are configured to fit about catheter 105. The holes can have a same diameter as catheter 105, a smaller diameter than catheter 105, or a larger diameter than catheter 105 depending on the embodiment. Balloon 120 can be mounted to catheter 105 at the point of entry and the point of exit using an adhesive, using one or more fasteners, and/or by any other suitable method such that an air-tight seal is formed between the holes in balloon 120 and catheter 105. In an alternative embodiment, catheter 105 may not extend all the way through balloon 120, and balloon 120 may be mounted to catheter 105 only at a point of entry of catheter 105. FIG. 1A illustrates balloon 120 in a deflated state. In the deflated state, balloon 120 can be substantially taut such that an external surface of balloon 120 is proximate to catheter 105.

Distal end 110 of catheter 105 also includes a distal needle port 150 through which a needle can be protruded and a distal balloon port 155 for inflating balloon 120. Distal needle port 150 can be a hole or opening through which a needle can be protruded. The needle can have a diameter of between approximately 1 mm and 2 mm, between approximately 0.5 mm and 3 mm, etc. depending on the size of the subject, the size of the cavity, etc. Distal balloon port 155 can be one or more holes or openings through which a fluid (i.e., a biocompatible gas or liquid as described in more detail below) can be introduced into balloon 120. Distal needle port 150 can be mounted to needle conduit 140 using an adhesive, one or more fasteners, etc. Distal needle port 150 may also be an extension of needle conduit 140. Needle conduit 140 can be cylindrical in shape and can be made from any suitable biocompatible material(s) known to those of skill in the art. Alternatively, needle conduit 140 can be any other suitable shape. In one embodiment, needle conduit 140 can be formed integrally as part of catheter 105, and can be made from the same material as catheter 105. Alternatively, needle conduit 140 can be formed separately from catheter 105 and assembled into catheter 105. Needle conduit 140 can be approximately the same length as catheter 105. The diameter of needle conduit 140 can be large enough to allow passage of the needle, but smaller than the overall diameter of catheter 105 less the diameter of balloon conduit 145. Needle conduit 140 can be uniform in diameter or may have a varying diameter. As an example, the diameter of needle conduit 140 may vary at distal needle port 150 and/or proximal needle port 130.

Distal needle port 150 may also be mounted to balloon 120 such that a needle can be protruded from distal needle port 150 without puncturing balloon 120. The needle can be any type of ostomy needle known to those of skill in the art. In an illustrative embodiment, distal needle port 150 is mounted in an air-tight fashion within a hole in a surface of balloon 120 such that the perimeter of the hole in the surface of balloon 120 forms an air tight seal about needle conduit 140. As such, balloon 120 can be inflated without fluid escaping through the hole in the surface of balloon 120. Distal needle port 150 can be mounted to balloon 120 using an adhesive, one or more fasteners, etc. Distal needle port 150 can be mounted such that an outer surface of needle port 150 is flush with the surface of balloon 120. Alternatively, the outer surface of needle port 150 may be mounted such that the outer surface protrudes outward from the surface of balloon 120. Distal needle port 150, which can be made from any suitable biocompatible material(s) known to those of skill in the art, can be an opening of any shape through which a needle can be protruded. Distal needle port 150 can have a diameter that allows protrusion of the needle through distal needle port 150. The diameter, which can be smaller than the diameter of catheter 105, can be the same as, larger than, or smaller than the diameter of needle conduit 140.

Proximal end 115 of catheter 105 includes a proximal needle port 130 for receiving the needle and a proximal balloon port 135 for receiving a fluid to inflate balloon 120. Alternatively, proximal needle port 130 may be replaced by a plunger mounted to the needle and used to control protrusion of the needle from distal needle port 150. Balloon 120 can be inflated within the cavity of the subject to position a cavity wall of the cavity adjacent to an abdominal wall of the subject. In an illustrative embodiment, balloon 120 can be inflated with a fluid that is introduced into proximal balloon port 135 of catheter system 100. The fluid can travel from proximal balloon port 135 through a balloon conduit 145 and out of distal balloon port 155 such that balloon 120 is inflated. In an alternative embodiment, a plurality of distal balloon ports may be used. The plurality of distal balloon ports can be a plurality of holes in balloon conduit 145 that are configured to introduce the fluid into balloon 120. The plurality of holes can be configured in a straight line along balloon conduit 145, on opposing sides of balloon conduit 145, etc. The fluid used to inflate balloon 120 can be air, inert gas, saline solution, water, and/or any other biocompatible gas or liquid. FIG. 1B illustrates catheter 105, needle conduit 140, and balloon conduit 145 in accordance with an illustrative embodiment. In alternative embodiments, other sizes of needle conduit 140 and/or balloon conduit 145 may be used. Other configurations of needle conduit 140 and/or balloon conduit 145 relative to catheter 105 may also be used.

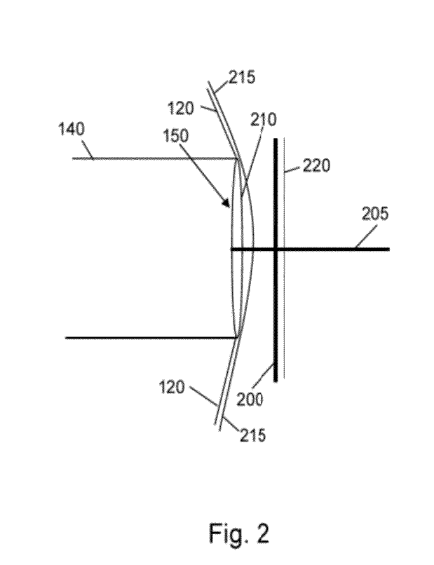

As illustrated with reference to FIG. 1C, as balloon 120 is inflated, distal needle port 150 and at least a portion of needle conduit 140 can bend and deflect away from a central axis 160 of catheter 105. As such, balloon 120 can be positioned to place distal needle port 150 in a desired orientation relative to the cavity wall and/or the abdominal wall of the subject. During the positioning process, balloon 120 may be at least partially deflated/re-inflated as appropriate. An illustrative orientation of distal needle port 150 is described in more detail with reference to FIG. 2. In an illustrative embodiment, balloon 120 and distal needle port 150 can be positioned within the cavity of the subject with the assistance of markers 125. Markers 125 can be made of radio-opaque material(s) such as barium sulfate or other metals and can be used in conjunction with x-ray radiation to position balloon 120 and distal needle port 150. As such, a physician, veterinarian, or other user can control the amount of inflation of balloon 120 based on a visual analysis of x-ray images. Alternatively, any other suitable material(s) and/or type of radiation may be used. Balloon 120 and distal needle port 150 can be positioned before, during, and/or after inflation of balloon 120. In alternative embodiments, fewer or additional markers 125 may be used and/or markers 125 may be placed on any other components of catheter system 100. For example, one or more markers 125 may be placed on a surface of balloon 120, on distal needle port 150, and/or on needle conduit 140. In one embodiment, needle conduit 140, distal needle port 150, and/or any other component of catheter system 100 may be made from radio-opaque material(s).

In an alternative embodiment, catheter system 100 may also include one or more magnets for use in positioning balloon 120 and/or distal needle port 150. The one or more magnets can be mounted to catheter 105, balloon 120, and/or distal needle port 150. A physician, veterinarian, or other user can place an external magnet on or near the surface of the subject's skin, and use the external magnet to move the one or more magnets mounted to catheter system 100 (which are within the cavity of the subject). The one or more magnets may be used alone and/or in conjunction with markers 125 to position distal needle port 150. In another alternative embodiment, one or more protuberances may be mounted to catheter 105, balloon 120, and/or to distal needle port 150. The one or more protuberances can be knobs, bulges, or any other types of protrusions. A physician can place his/her hand on the subject's outer skin to locate and manipulate the one or more protuberances within the cavity such that distal needle port 150 can be positioned. The one or more protuberances may also be externally visible as one or more bulges in the subject's outer skin. The one or more protuberances may be used alone and/or in conjunction with markers 125 to position distal needle port 150. Further, any of the methods described herein for positioning balloon 120 and/or distal needle port 150 may be used in any combination.

In another alternative embodiment, different portions of balloon 120 may be of different thicknesses such that balloon 120 is substantially hemispherical in shape when inflated. As an example, material used to form a base of balloon 120 may be thicker than material used to form a hemisphere portion of balloon 120. As such, when balloon 120 is inflated, the base portion of balloon 120 can remain a substantially flat surface that does not significantly expand outward due to the thicker material used to form the base portion. The hemisphere portion of balloon 120, which is formed from thinner, more elastic material than the base portion, can expand to form a substantially hemispherical or dome shape, a base of which is formed by the substantially flat base portion of balloon 120. Alternatively, different types of materials having the same or different thicknesses may be used to form the base and the hemisphere portions of balloon 120. In yet another alternative embodiment, balloon 120 may be mounted to one side of the catheter instead of surrounding the catheter such that balloon 120 is substantially hemispherical in shape when inflated. In an illustrative embodiment, distal needle port 150 can be mounted to the hemisphere portion of balloon 120.

Once balloon 120 and distal needle port 150 are positioned, a needle can be protruded through distal needle port 150 to place a hole in the cavity wall, the abdominal wall, and the outer skin of the subject. The needle can be protruded between approximately 1 cm to 30 cm, between approximately 5 cm and 25 cm, between approximately 10 cm and 20 cm, etc. past the outer skin of the subject. The physician, veterinarian, or other user can protrude any length of the needle through the outer skin of the subject, depending on the procedure. The needle, which can have a sharp distal tip for the purpose of penetrating tissue, can be made from a biocompatible metal or other biocompatible material. In one embodiment, the needle can have a length that is equal to or greater than the length of catheter 105. The needle can be inserted into proximal needle port 130, through needle conduit 140, and protruded from distal needle port 150 after balloon 120 and distal needle port 150 are positioned within the cavity of the subject. In one embodiment, the needle may be mounted to a wire or other semi-rigid structure such that the needle can be protruded through the various tissue layers of the subject. A user can manipulate the wire or other semi-rigid structure to protrude and retract the needle. The wire or other semi-rigid structure can be made from a biocompatible metal or other biocompatible material. In such an embodiment, the length of the needle may be between approximately 1 cm and 10 cm, between approximately 3 cm and 6 cm, approximately 5 cm, etc. In an alternative embodiment, the needle may be mounted within needle conduit 140. In such an embodiment, a plunger may be mounted in place of or in addition to proximal needle port 130 to protrude the needle outward from distal needle port 150 and through the tissue layers. Once the needle is protruded outward from the cavity and through the subject's outer skin, a medical tube can be placed through the hole and into the cavity and secured using any methods known to those of skill in the art. Alternatively, the medical tube may not be placed. The medical tube can be a feeding tube, a drainage tube, etc. Upon placement of the medical tube, the needle can be withdrawn, balloon 120 can be deflated by withdrawing the fluid through balloon conduit 145 and out of proximal balloon port 135, and catheter system 100 can be removed from the subject. Balloon 120 can be deflated by withdrawing all of the fluid from balloon 120. Alternatively, only a portion of the fluid may be withdrawn until balloon 120 is small enough to fit through the opening by which catheter system 100 was inserted into the subject.

In an alternative embodiment, once the needle is protruded through the outer skin of the subject, the medical tube may be inserted through the mouth or other opening through which catheter system 100 is inserted into the cavity. In such an embodiment, the needle can be used as a guide wire and the medical tube can pass over the needle and out through the subject. For placement, the medical tube can pass through needle conduit 140 such that the needle and any control wire, etc. attached to the needle is within an interior of the medical tube. Alternatively, once in place, the needle may be detached from catheter system 100 such that catheter system 100 can be removed from the cavity. The needle, which may be secured by a human hand, an anchor, etc., can act as a guide wire for a medical tube introduced through the mouth or other opening through which catheter system 100 was inserted.

FIG. 2 illustrates an orientation of distal needle port 150 relative to a portion of an abdominal wall 200 in accordance with an illustrative embodiment. In alternative embodiments, any other suitable orientation may be used. In FIG. 2, a needle 205 is suitably positioned to penetrate an external surface (or outer skin 220) of a subject. As used herein, suitably positioned can refer to needle 205 protruding out from distal needle port 150 at approximately a ninety degree angle relative to a plane that contains an outer surface 210 of distal needle port 150. As used herein, approximately a ninety degree angle can refer to any angle between forty-five degrees and one hundred thirty-five degrees. Alternatively, any other suitable angle may be used. As needle 205 is protruded from distal needle port 150, needle 205 punctures and places a hole in a cavity wall 215, the portion of abdominal wall 200, and outer skin 220 of the subject. Needle 205 punctures the portion of abdominal wall 200 of the subject at approximately a ninety degree angle. Suitably positioning needle 205 can be also be performed by positioning the plane that contains outer surface 210 of distal needle port 150 substantially parallel to the portion of abdominal wall 200 such that needle 205 punctures abdominal wall 200 at approximately the same angle at which needle 205 protrudes from distal needle port 150. In embodiments in which outer surface 210 is not flat (i.e., it may be rounded, beveled, etc.), the plane can contain at least a portion of outer surface 210. As used herein, substantially parallel can refer to any orientation within forty-five degrees of parallel. Alternatively, any other suitable angle may be used.

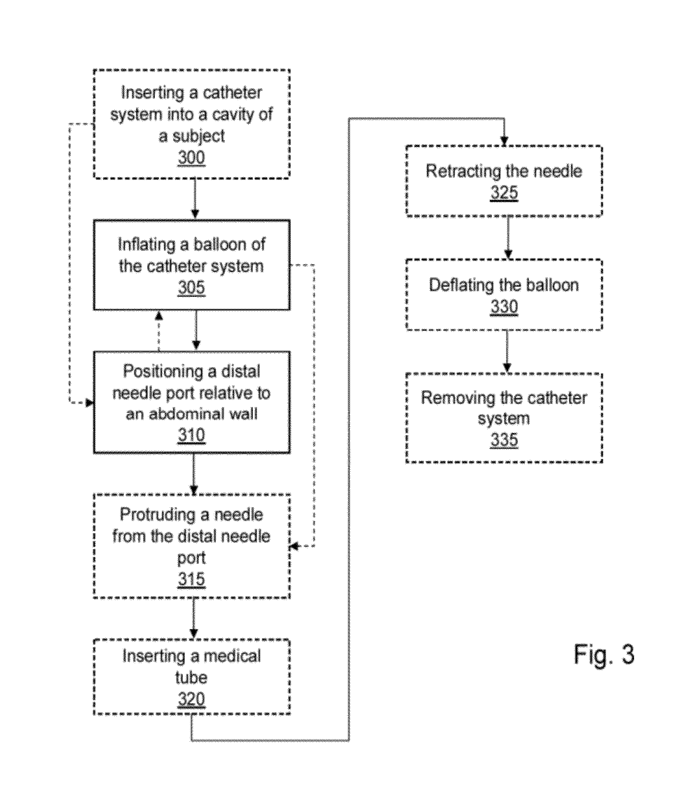

FIG. 3 is a flow diagram illustrating a process for performing a transluminal ostomy in accordance with an illustrative embodiment. Dashed boxes are used in FIG. 3 to identify optional operations. The ostomy can be a gastrostomy, jejunostomy, etc. A catheter system is inserted into a cavity of a subject in an operation 300. The catheter system can be catheter system 100 described with reference to FIGS. 1A-1C and 2. In an illustrative embodiment, the catheter system can be introduced through a mouth or other opening of the subject, and the cavity can be a stomach cavity, a jejunal lumen, a bladder cavity, etc. A distal end of the catheter system can be positioned within the cavity. The catheter system can be inserted with the assistance of a guide wire as described with reference to FIG. 1. Alternatively, a physician, veterinarian, or other user may position the catheter system based on a length of catheter that is inserted through the mouth or other opening. The physician, veterinarian, or other user may also place his/her hands on the stomach area of the subject to feel the catheter system underneath the subject's skin and help guide the catheter system into the cavity. In one embodiment, the physician, veterinarian, or other user can use x-ray radiation along with radio-opaque markers on the catheter system to position the catheter system within the cavity.

A balloon mounted to the distal end of the catheter system is inflated in an operation 305. The balloon can be inflated by introduction of a fluid into the balloon by way of one or more ports as described with reference to FIGS. 1A-1C. In an illustrative embodiment, a distal needle port mounted to the balloon can deflect and bend away from a central axis of the catheter as the balloon is inflated. The distal needle port can be mounted to a needle conduit for receiving a needle. Inflation of the balloon can cause a cavity wall of the cavity to expand and become adjacent to an abdominal wall of the subject. In one embodiment, inflation of the balloon can cause the cavity wall to touch the abdominal wall. However, depending on the subject, inflation of the balloon may only place the cavity wall within one or more millimeters of the abdominal wall, within one or more centimeters of the abdominal wall, etc. Positioning the cavity wall adjacent to the abdominal wall can refer to the cavity wall touching the abdominal wall or not touching the abdominal wall.

The distal needle port is positioned relative to the abdominal wall of the subject in an operation 310. As illustrated by the dashed arrows in FIG. 3, the distal needle port can be positioned before, during, or after inflation of the balloon, depending on the embodiment. In an illustrative embodiment, the distal needle port can be positioned such that a needle protruded from the distal needle port at an angle of approximately ninety degrees punctures the abdominal wall at approximately the ninety degree angle. Alternatively, any other suitable orientation may be used. The distal needle port may be positioned using one or more radio-opaque markers in conjunction with x-ray radiation. As an example, the physician, veterinarian, or other user may monitor the one or more radio-opaque markers within the cavity using X-ray, for example. The catheter system can be adjusted based on the positions of the radio-opaque markers, a known orientation of the radio-opaque markers relative to the distal needle port, and a position of the abdominal wall. In an alternative embodiment, x-ray radiation may not be used. In one embodiment, one or more magnets and/or one or more protuberances mounted to the catheter system may be used to position the distal needle port.

A needle is protruded from the distal needle port in an operation 315. The needle, which can be mounted to a wire or other semi-rigid structure, can be inserted into a proximal needle port of the catheter system, through the needle conduit, and out from the distal needle port. As such, the needle can puncture the cavity wall, the abdominal wall, and the outer skin of the subject to protrude outward from the subject's abdominal area. In an alternative embodiment, the needle may be mounted within the needle conduit and plunged through the various layers of tissue (i.e., cavity wall, abdominal wall, and skin) once the distal needle port is in a desired position relative to the abdominal wall.

A medical tube is inserted in an operation 320. In one embodiment, the hole may be enlarged prior to insertion of the medical tube using any procedures known to those of skill in the art. In an illustrative embodiment, the medical tube may be inserted (from outside of the subject) into the hole and into the cavity. Alternatively, the medical tube may be inserted into the cavity through the mouth or other opening of the subject and out through the hole. The medical tube can be a feeding tube, a drainage tube, etc. The needle is retracted in an operation 325, and the balloon is deflated in an operation 330. In an illustrative embodiment, deflation of the balloon can cause the balloon and the distal needle port to compress about the catheter. The catheter system is removed from the cavity of the subject in an operation 335. Alternatively, operation 325 may be omitted and the needle may be removed along with the catheter system in operation 335. Alternatively, an ostomy may be performed and a medical tube may not be inserted into the subject.

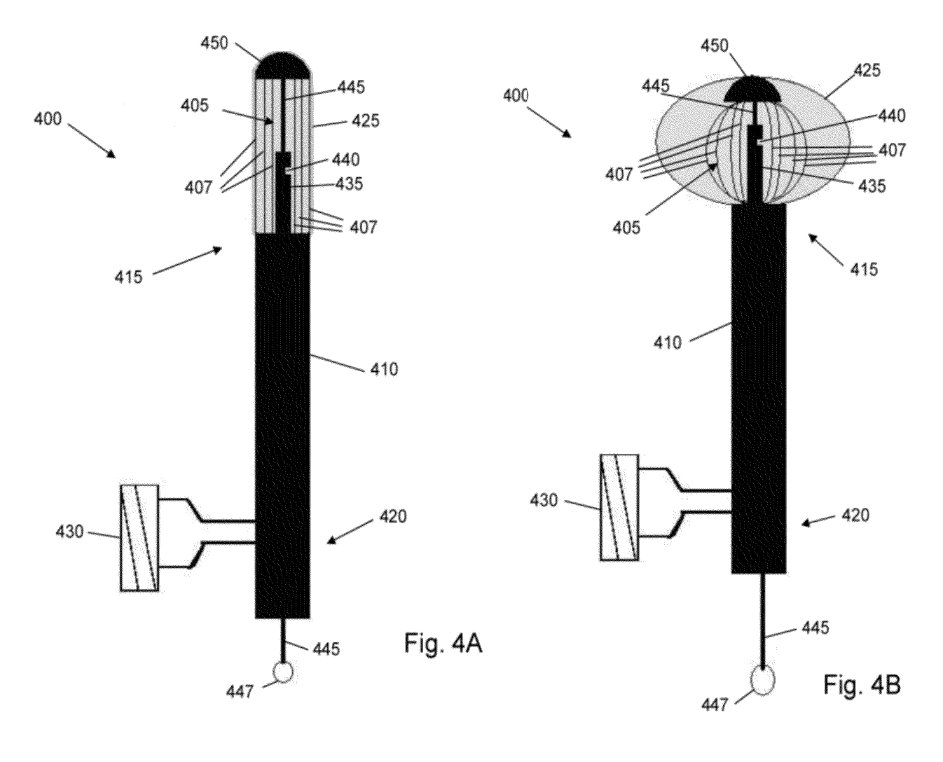

FIG. 4A is a side view of a catheter system 400 with a stent 405 in a compressed state in accordance with an illustrative embodiment. FIG. 4B is a side view of catheter system 400 with stent 405 in an expanded state in accordance with an illustrative embodiment. In one embodiment, catheter system 400 can have approximately the same dimensions and/or be made of the same materials as catheter system 100 described with reference to FIGS. 1A-1C, 2, and 3. Alternatively, any other dimensions and/or suitable materials may be used.

Catheter system 400 includes a catheter 410 having a distal end 415 and a proximal end 420. A balloon 425 is mounted to distal end 415 such that stent 405 is within an interior of balloon 425. In an alternative embodiment, balloon 425 may not be included, and catheter system 400 may use stent 405 to provide all tissue support. In use, distal end 415 of catheter system 400 can be placed into a cavity of a subject by way of the mouth or other opening while balloon 425 is deflated and stent 405 is compressed as illustrated in FIG. 4A. The cavity can be a stomach (or gastric cavity), jejunal lumen, intestine, bladder, and/or any other applicable cavity. Catheter system 400 can be inserted using a guide wire as described above with reference to FIG. 1. In such an embodiment, catheter system 400 may include a guide wire conduit and one or more guide wire ports for receiving the guide wire. Alternatively, catheter system 400 may be placed based on visual observation of a length of catheter 410 that remains outside of the subject, touching of the abdominal wall to feel catheter system 400, etc. One or more radio-opaque markers, protuberances, and/or magnets may also be mounted to catheter system 400 for use in positioning catheter system 400 as described with reference to FIGS. 1A-1C and 3.

Once catheter system 400 is positioned within the cavity, balloon 425 can be inflated by introducing a fluid into a proximal balloon port 430, through a balloon conduit 435, and out of a distal balloon port 440 into the interior of balloon 425. The fluid can be air, inert gas, saline solution, water, and/or any other biocompatible gas or liquid as described above. Inflation of balloon 425 can cause a cavity wall of the cavity to become adjacent to an abdominal wall of the subject. Positioning the cavity wall adjacent to the abdominal wall can refer to the cavity wall touching the abdominal wall or not touching the abdominal wall, depending on the subject, etc.

As illustrated in FIGS. 4A and 4B, stent 405 includes a plurality of flexible wires 407. In alternative embodiments, additional or fewer wires 407 may be included. Wires 407 can be made from a biocompatible metal or other biocompatible material used for stents. One end of each of wires 407 can be mounted to a cap 450 and/or to a center wire 445, and the other end of each of wires 407 can be mounted to catheter 410 or balloon conduit 435. The lengths and/or diameters of the stent 405 may be determined based on the size of the subject, the size of the cavity, etc. In addition, any other suitable stent configuration may be used. Stent 405 can be expanded using center wire 445 of stent 405. Center wire 445 can be more rigid that wires 407 and can have a larger diameter than wires 407. Center wire 445 can be made from the same material as wires 407 or from a different material, depending on the embodiment. Center wire 445, which is mounted to cap 450 of stent 405, can be pulled outward from proximal end 420 of catheter 410 (by a user) to draw cap 450 towards catheter 410 and expand stent 405. Stent 405 is expanded when wires 407 bend or bulge outward as illustrated in FIG. 4B. The user can use a center wire handle 447 to pull center wire 445 outward to expand stent 405 and to push center wire 445 inward to compress stent 405 by causing wires 407 to straighten as illustrated in FIG. 4A. The expanded diameter of the stent may also be controlled by adjusting the position of the center wire. Stent 405 can be expanded before, during, or after inflation of balloon 425, depending on the embodiment. In one embodiment, center wire 445 can be partially housed in a stent conduit (not shown) that runs along an interior of catheter 410. Alternatively, center wire 445 may run through balloon conduit 435. In such an embodiment, points of entry and/or exit of center wire 445 into and/or from balloon conduit 435 can be sealed such that balloon 425 can be inflated with the fluid. In an illustrative embodiment, stent 405, when fully expanded, can be substantially the same size as balloon 425 (when inflated). In alternative embodiments, any of the conduits can be combined into one or more conduits.

Once balloon 425 is inflated and stent 405 is expanded within the cavity of the subject, a physician, veterinarian, or other user can insert a needle percutaneously through outer skin of the subject, through the abdominal wall, through the cavity wall, through balloon 425, and into the cavity of the subject. In an illustrative embodiment, the needle may be inserted such that the needle enters both the abdominal wall and the cavity wall at an angle of approximately ninety degrees. Alternatively, any other suitable angle may be used. As the needle punctures balloon 425, balloon 425 may begin to deflate. As a result, balloon 425 may be unable to maintain the position of the cavity wall relative to the abdominal wall. However, stent 405, which is in the expanded state, can prevent the cavity wall from collapsing and can substantially maintain a position of the cavity wall relative to the abdominal wall. As such, stent 405 can help prevent the needle from slipping out of the cavity and also help prevent the needle from undesirably puncturing a posterior cavity wall.

Once the needle is in place and optionally secured (e.g., using an anchor or any other method known to those of skill in the art), a medical tube can be inserted into the cavity using any procedures known to those of skill in the art. Stent 405 can be compressed by pushing center wire 445 into proximal end 420 of catheter 410 such that cap 450 is pushed outward from distal end 415 of catheter 410. The user can use center wire handle 447 to push center wire 445 into catheter 410 to compress stent 405. Catheter system 400 can be withdrawn from the cavity of the subject to complete the procedure. Alternatively, a medical tube may not be placed into the cavity.

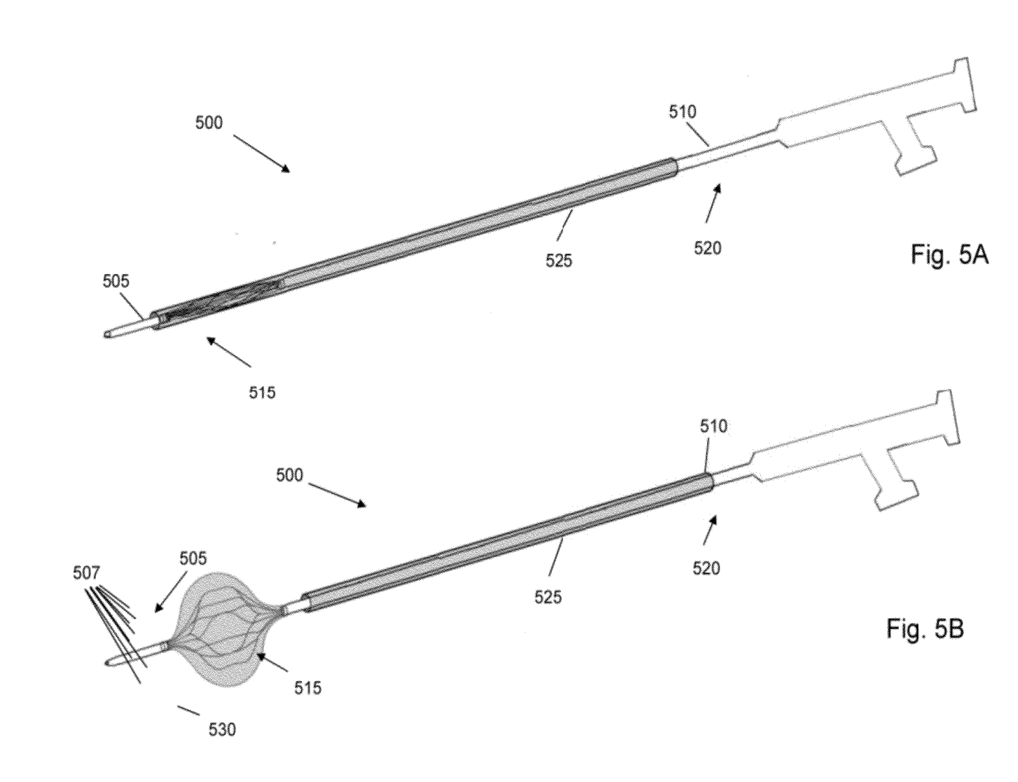

FIG. 5A illustrates a catheter system 500 with a stent 505 in a compressed state in accordance with a second illustrative embodiment. FIG. 5B illustrates catheter system 500 with stent 505 in an expanded state in accordance with an illustrative embodiment. With the exception of expansion and compression of stent 505, the use and function of catheter system 500, including balloon configuration, can be similar to that of catheter system 400 described with reference to FIGS. 4A and 4B, and will not be described in detail. Catheter system 500 includes a catheter 510 having a distal end 515 and a proximal end 520. A sheath 525 is mounted to catheter 510. Sheath 525 (or sleeve) can be made from a biocompatible material. Sheath 525 can be shorter in length than catheter 510 and can have a diameter larger than catheter 510. The interior diameter of sheath 525 can be large enough such that sheath 525 can maintain stent 505 in a compressed state as illustrated in FIG. 5A. Sheath 525 can be mounted to catheter 510 by any method known to those of skill in the art.

In FIG. 5A, sheath 525 is positioned over stent 505 and a balloon 530 that surrounds stent 505 such that stent 505 is maintained in a compressed state. Stent 505 includes a plurality of flexible wires 507 that are configured to compress when sheath 525 is positioned over wires 507 (as illustrated in FIG. 5A) and expand when sheath 525 is retracted from wires 507 (as illustrated in FIG. 5B). Distal end 515 of catheter 510 can be placed into a cavity of a subject while stent 505 is in the compressed state. A physician, veterinarian, or other user can slide sheath 525 along catheter 510 in a direction away from distal end 515 and toward proximal end 520. Stent 505 can expand as sheath 525 is withdrawn. The user can withdraw sheath 525 using his/her hands or by any other suitable means. Balloon 530 can be inflated, a needle can be inserted through the subject's skin and into the cavity, and stent 505 can prevent the cavity wall from collapsing as balloon 530 deflates as a result of being punctured. A medical tube can be placed into the cavity as described above with reference to FIGS. 4A and 4B. Alternatively, a medical tube may not be placed into the cavity. The physician, veterinarian, or other user can slide sheath 525 along catheter 510 in a direction away from proximal end 520 and toward distal end 515 to compress stent 505 under sheath 525. The physician, veterinarian, or other user can remove catheter system 500 with stent 505 in the compressed state.

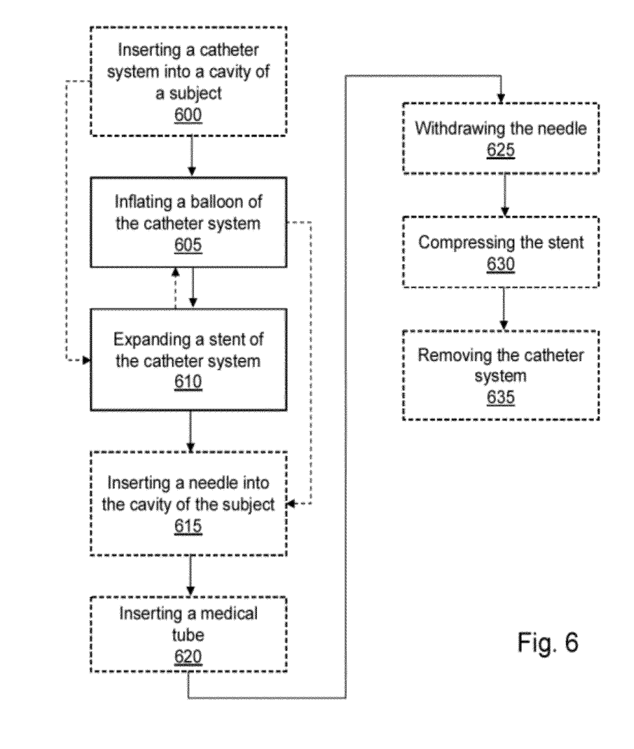

FIG. 6 is a flow diagram illustrating a process for performing a percutaneous ostomy in accordance with an illustrative embodiment. Dashed boxes are used in FIG. 6 to identify optional operations. The ostomy can be a gastrostomy, jejunostomy, etc. A catheter system is inserted into a cavity of a subject in an operation 600. In an illustrative embodiment, the catheter system can be catheter system 400 described with reference to FIGS. 4A and 4B and/or catheter system 500 described with reference to FIGS. 5A and 5B. In another illustrative embodiment, the catheter system can be introduced through a mouth and esophagus (if present) of the subject, and the cavity can be a stomach cavity or a jejunal lumen. A distal end of the catheter system can be positioned within the cavity. The catheter system can be inserted with the assistance of a guide wire as described with reference to FIG. 1. Alternatively, a physician, veterinarian, or other user may position the catheter system based on a length of catheter that is inserted through the mouth. The physician, veterinarian, or other user may also place his/her hands on the stomach area of the subject to feel the catheter system underneath the subject's skin and help guide the catheter system into the cavity. In one embodiment, the physician, veterinarian, or other user can use x-ray radiation along with radio-opaque markers on the catheter system to position the catheter system within the cavity.

A balloon mounted to the distal end of the catheter system is inflated in an operation 605. The balloon can be inflated by introduction of a fluid into the balloon by way of one or more ports. Inflation of the balloon can cause a cavity wall of the cavity to expand and become adjacent to an abdominal wall of the subject. In one embodiment, inflation of the balloon can cause the cavity wall to touch the abdominal wall. However, depending on the subject, inflation of the balloon may only place the cavity wall within one or more millimeters of the abdominal wall, within one or more centimeters of the abdominal wall, etc. Positioning the cavity wall adjacent to the abdominal wall can refer to the cavity wall touching the abdominal wall or not touching the abdominal wall.

A stent of the catheter system is expanded in an operation 610. In an illustrative embodiment, the stent is within an interior of the balloon. As illustrated by the dashed arrows in FIG. 6, the stent can be expanded before, during, or after inflation of the balloon, depending on the embodiment. The stent can be expanded by manipulating a center wire of the stent, by adjusting a position of a sheath mounted to the catheter system, and/or by any other suitable method. A physician, veterinarian, or other user percutaneously inserts a needle into the cavity of the subject in an operation 615. The needle can be inserted through the external skin of the subject, through the abdominal wall of the subject, through the cavity wall of the subject, through the balloon, and into the cavity of the subject. In an illustrative embodiment, the needle may be inserted such that the needle enters both the abdominal wall and the cavity wall at an angle of approximately ninety degrees. Alternatively, any other suitable angle may be used. As the balloon deflates as a result of being punctured, the stent can prevent the cavity wall from collapsing and can substantially maintain the position of the cavity wall relative to the abdominal wall.

A medical tube is inserted in an operation 620. In one embodiment, the hole may be enlarged prior to insertion of the medical tube using any procedures known to those of skill in the art. The medical tube can be a feeding tube, a drainage tube, etc. In an illustrative embodiment, the medical tube may be inserted (from outside of the subject) into the hole and into the cavity. Alternatively, the medical tube may be inserted into the cavity through the mouth or other opening of the subject and out through the hole. The needle is withdrawn in an operation 625, and the stent is compressed in an operation 630. The stent can be compressed by manipulating a center wire of the stent, by adjusting a position of a sheath mounted to the catheter system, and/or by any other suitable method. The catheter system is removed from the cavity of the subject in an operation 635. Alternatively, an ostomy may be performed and a medical tube may not be inserted into the subject.

One or more flow diagrams have been used herein. The use of flow diagrams is not meant to be limiting with respect to the order of operations performed. The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A system comprising:
a catheter comprising:
a first conduit having a first distal port, wherein the first distal port is in fluid communication with an interior of a balloon mounted to the catheter; and
a second conduit having a second distal port, wherein the second distal port is configured to receive a needle; and
the balloon, wherein the balloon is configured to inflate responsive to a fluid received through the first distal port such that the needle is suitably positioned to penetrate an external surface of a subject.

2. The system of claim 1, wherein the catheter further comprises a third conduit configured to receive a guide wire.

3. The system of claim 1, wherein the second distal port is mounted to the balloon, and wherein the inflation of the balloon causes the second distal port to bend away from a central axis of the catheter.

4. The system of claim 1, wherein the inflation of the balloon positions the second distal port such that the needle enters a portion of an abdominal wall of the subject at approximately a ninety degree angle.

5. The system of claim 1, wherein the catheter further comprises one or more radio-opaque markers for use in conjunction with X-ray radiation to position the catheter within the subject.

6. The system of claim 1, wherein the catheter further comprises one or more radio-opaque markers for use in conjunction with X-ray radiation to position the second distal port relative to a portion of an abdominal wall of the subject.

7. The system of claim 1, further comprising a protuberance mounted to at least one of the balloon or the second distal port, wherein the protuberance is configured to provide an indication of a position of the second distal port.

8. The system of claim 1, further comprising a magnet mounted to at least one of the balloon or the second distal port, wherein the magnet is used to position the second distal port.

9. The system of claim 1, wherein a first portion of the balloon has a first thickness and a second portion of the balloon has a second thickness to form a substantially hemispherical shape comprising a base and a hemisphere when the balloon is inflated, and wherein the second distal port is mounted to at least a portion of the hemisphere.

10. A method comprising:
inserting a catheter into a cavity of a subject, wherein the catheter comprises a first conduit having a first distal port in fluid communication with an interior of a balloon mounted to the catheter, and a second conduit having a second distal port;
inflating the balloon to suitably position a needle to penetrate an external surface of the subject; and
protruding the needle through the second distal port to puncture the external surface of the subject.

11. The method of claim 10, wherein the cavity comprises at least one of a gastric cavity or a jejunal lumen.

12. The method of claim 10, further comprising:
identifying positions of one or more radio-opaque markers with X-ray radiation, wherein the one or more radio-opaque markers are mounted to at least one of the catheter, the balloon, or the second distal port; and
adjusting a position of the second distal port based at least in part on the positions of the one or more radio-opaque markers.

13. The method of claim 10, wherein inflating the balloon causes the second needle port and at least a portion of the second conduit to bend away from a central axis of the catheter.

14. A system comprising:
a catheter comprising a first conduit having a first distal port; and
a stent mounted to the catheter so that the stent is within an interior of a balloon mounted to the catheter, wherein the stent is expandable to maintain a position of a cavity wall of a subject after the balloon at least partially deflates.

15. The catheter system of claim 14, wherein the cavity wall comprises at least one of a stomach wall or an intestinal wall.

16. The catheter system of claim 14, wherein the stent comprises a center wire, one or more flexible wires, and a cap, and wherein the center wire is mounted to at least the cap for movement of the cap.

17. The catheter system of claim 16, wherein movement of the cap toward a proximal end of the catheter expands the one or more flexible wires of the stent, and wherein movement of the cap away from the proximal end of the catheter compresses the one or more flexible wires of the stent.

18. The catheter system of claim 14, wherein the stent comprises one or more flexible wires, and further comprising a sheath surrounding at least a portion of the catheter, wherein movement of the sheath toward a proximal end of the catheter expands the one or more flexible wires of the stent, and wherein movement of the sheath away from the proximal end of the catheter compresses the one or more flexible wires of the stent.

19. The catheter system of claim 14, wherein at least one of the catheter, the balloon, or the stent comprises one or more radio-opaque markers for use in conjunction with X-ray radiation to position the catheter within the subject.

20. The catheter system of claim 14, further comprising the balloon, wherein the first distal port is in fluid communication with the interior of the balloon to inflate the balloon, and wherein inflation of the balloon positions a cavity wall of a subject adjacent to an abdominal wall of the subject.
